SEQUENCE LISTING (2 sequences)

SEQ ID NO: 1 - protein, 15 aa, artificial sequence
  peanut peptide
WELQGDRRCQSQLER

SEQ ID NO: 2 - protein, 15 aa, artificial sequence
  peanut peptide
DSYERDPYSPSQDPY

(19) United States
(12) Patent Application Publication
(10) Pub. No.: US 2021/0247404 A1
(43) Pub. Date: Aug. 12, 2021

(54) Assays For Detecting Peanut Allergies

(71) Applicant: AllerGenis LLC, Hatfield, PA (US)

(72) Inventors: Robert C. Getts, Hatfield, PA (US); Paul Kearney, Hatfield, PA (US)

(21) Appl. No.: 17/168,350

(22) Filed: Feb. 5, 2021

Related U.S. Application Data
(60) Provisional application No. 62/970,822, filed on Feb. 6, 2020.

(51) Int. Cl. G01N 33/68; A61K 39/35; C07K 14/415

(52) U.S. Cl. CPC G01N 33/6854; A61K 39/35; A61K 2039/55; G01N 2800/24; G01N 2333/415; C07K 14/415

(57) ABSTRACT

The present disclosure provides peanut peptide compositions and kits, and methods for diagnosis of peanut allergy, methods for detecting the development of clinical tolerance to peanuts, and methods for desensitizing an infant to peanut allergens.

### FIELD

The present disclosure is directed, in part, to peanut peptide compositions and kits, and methods for diagnosis of peanut allergy, methods for detecting the development of clinical tolerance to peanuts, and methods for desensitizing an infant to peanut allergens.

### BACKGROUND

Food allergies are a common problem among adults and children, and symptoms may range from mild oral pruritus to potentially life-threatening anaphylactic shock. Food allergies are currently diagnosed by skin prick testing or oral provocation, and measurement of serum levels of specific IgE and, in some cases, other serum antibodies, such as IgG4. Although these tests indicate the likelihood of clinical reactivity, they do not distinguish the different phenotypes of food allergy or provide prognostic information. Current allergy tests also involve some level of risk to the patient. The relationship between current IgE testing and the actual clinical sensitivity of the patient is a weak one that is usually defined as a combination of reaction severity and the amount of allergen that provokes a reaction. Another limitation of current testing is the inability to determine whether or not pediatric patients will outgrow the allergy during childhood. In this case there is a positive but weak correlation between specific IgE level and the duration of clinical allergy.

More recently, it has been suggested that clinical reactivity to food allergens may correlate better with allergen-specific IgE on the epitope recognition level. It has been reported that patients with persistent or more severe allergic reactions recognize larger numbers of IgE epitopes, suggesting epitope mapping as an additional tool for allergy diagnosis and prediction. Spot membrane-based immunoassays have been used for epitope mapping. In this system, peptides are synthesized on the membrane and incubated with the patient's sera. The process requires a large number of peptides and is, therefore, error prone, time consuming, labor intensive, and expensive. Immunoassays in this format also require a large volume of patient serum.

The marked heterogeneity of clinical presentations for food allergy poses a challenge to successful management and treatment, and therefore precision medicine strategies are highly relevant to improve prevention, manage current cases and initiate new therapy in food allergy. Sensitive and specific biomarkers for determination of food allergy endotypes, risk of developing allergies, reaction severity, and prognosis with treatment are essential components in the path toward precision medicine (Sicherer et al., J. Allergy Clin. Immunol., 2015, 135, 357-67). In the past decade, there have been a number of studies evaluating the efficacy of oral immunotherapy (OIT) for the treatment of persistent food allergies (Wood et al., J. Allergy Clin. Immunol., 2016, 137, 1103-1110). In peanut allergy, OIT has been shown to have acceptable safety profile and demonstrated clinical benefit (Bird et al., J. Allergy Clin. Immunol. Pract., 2017, 5, 335-344). Despite the improvement in clinical reactivity, OIT has been associated with significant adverse effects, with some experiencing anaphylaxis and 15% to 20% forced to discontinue therapy because of adverse reactions (Bird et al., J. Allergy Clin. Immunol. Pract., 2017; Keet Et al., J. Allergy Clin. Immunol., 2012, 129, 448-455; Longo et al., J. Allergy Clin. Immunol., 2008, 121, 343-7; Meglio et al., Pediatr. Allergy Immunol., 2008, 19, 412-419; Skripak et al., J. Allergy Clin. Immunol., 2008, 122, 1154-60; Staden et al., Allergy, 2007, 62, 1261-1269). In addition to adverse reactions, the response to OIT is typically not sustained once therapy is discontinued, i.e. patients are temporarily desensitized to allergens but do not achieve tolerance (Wood et al., J. Allergy Clin. Immunol., 2016, 137, 1103-1110; Burks et al., N. Engl. J. Med., 2012, 367, 233-243; Burks et al., J. Allergy Clin. Immunol., 2008, 121, 1344-1350; Burks, Arb. Paul Ehrlich Inst. Bundesinstitut Impfstoffe Biomed Arzneim Langen Hess, 2013, 97, 122-123; Gorelik et al., J. Allergy Clin. Immunol., 2015, 135, 1283-1292; and Keet et al., J. Allergy Clin. Immunol., 2013, 132, 737-739). However, it is clear that progress is being made and new food allergy therapies are close to FDA approval. These therapeutic approaches will benefit from a diagnostic and prognostic test which will help patients and their doctors understand the severity of the disease upon entry into therapy, monitor a patient while on therapy to assess progress or onset of an adverse reaction before it occurs, and track patient status once treatment is discontinued.

The production of IgE antibodies against peanut proteins is central to the pathogenesis of peanut allergy. Although predictive curves have been generated to identify peanut specific IgE concentrations which are 95% predictive of clinical reactivity, peanut-IgE is poorly predictive at lower IgE levels, and at higher levels the readout is only binary and is therefore difficult to use to help assess the safety or efficacy of therapy. This may be due to measurement of IgE antibodies against components of peanut which are not clinically relevant. IgE against Ara h 2 predicts clinical reactivity to peanut (Lieberman et al., J. Allergy Clin. Immunol. Pract., 2013, 1, 75-82) but there is a great deal of clinical heterogeneity across individuals with similar levels of Ara h 2. Peptide microarrays comprised of overlapping peptides covering the entire sequential epitope repertoire of major allergens have been developed to measure the epitope-specific immunoglobulin response (Lin et al., J. Allergy Clin. Immunol., 2009, 124, 315-22; and Lin et al., J. Allergy Clin. Immunol., 2012, 129, 1321-1328). The number of peanut epitopes in Ara h 1, 2 and 3 which bind to IgE is predictive of reaction severity (Flinterman et al., J. Allergy Clin. Immunol., 2008, 121, 737-743). As a component-resolved diagnostic methodology (ImmunoCAP), the presence of sIgE to peanut, Ara h1, Ara h2, and Ara h3 is indicative of a “true” peanut allergy and a high risk of severe reactions (e.g., levels of sIgE≥0.35 kUA/L show 75-95% PPV, 90% NPV in diagnosing allergy; Klemans et al., J. Allergy Clin. Immunol., 2013, 131, 157-163).

### SUMMARY

The present disclosure provides methods for diagnosing a peanut allergy in a subject comprising: contacting two peanut peptides coupled to a solid support with a biological sample obtained from the subject under conditions sufficient to permit binding of one or more allergy associated immunoglobulins (AAIs) in the biological sample to the two peanut peptides to form AAI-peptide-solid support complexes, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein; contacting the AAI-peptide-solid support complexes with an AAI-specific labeling reagent to form labeling reagent-AAI-peptide-solid support complexes; and measuring the binding of the AAI-specific labeling reagent to each AAI-peptide-solid support complex; wherein when the combined binding for each peanut peptide in the AAI-peptide solid support complex to the AAI-specific labeling reagent is greater than a threshold value, the subject is allergic to peanuts, and when the combined binding for each peanut peptide in the AAI-peptide solid support complex to the AAI-specific labeling reagent is equal to or less than the threshold value, the subject is not allergic to peanuts.

The present disclosure also provides methods for detecting development of clinical tolerance to peanuts in a subject that is allergic to peanuts comprising: contacting two peanut peptides coupled to a solid support with a biological sample obtained from the subject under conditions sufficient to permit binding of one or more allergy associated immunoglobulins (AAIs) in the biological sample to the two peanut peptides to form two AAI-peptide-solid support complexes, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein; contacting the AAI-peptide-solid support complexes with an AAI-specific labeling reagent to form labeling reagent-AAI-peptide-solid support complexes; measuring the binding of the AAI-specific labeling reagent to each AAI-peptide-solid support complex; and comparing the binding of the AAI-specific labeling reagent to each AAI-peptide-solid support complex to the binding of the AAI-specific labeling reagent to each AAI-peptide-solid support complex from a biological sample previously obtained from the subject; wherein when the combined binding for each peanut peptide in the AAI-peptide solid support complex to the AAI-specific labeling reagent is greater than or equal to the combined binding for each peanut peptide in the AAI-peptide solid support complex to the AAI-specific labeling reagent for the previously obtained biological sample, the subject has not established clinical tolerance to peanuts; and when the combined binding for each peanut peptide in the AAI-peptide solid support complex to the AAI-specific labeling reagent is less than the combined binding for each peanut peptide in the AAI-peptide solid support complex to the AAI-specific labeling reagent for the previously obtained biological sample, the subject has established clinical tolerance to peanuts.

The present disclosure also provides methods of desensitizing an infant to two peanut allergens to induce tolerance or non-allergy to peanuts comprising administering two peanut peptides to the infant, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein.

The present disclosure also provides compositions consisting of a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein.

The present disclosure also provides kits comprising: a solid support coupled to a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein; and an allergy associated immunoglobulin (AAI)-specific labeling reagent.

### BRIEF DESCRIPTION OF THE DRAWINGS

FIG. 1 shows results of a test on the validation cohort, as well as performance of other diagnostic tests displayed for comparison.

### DESCRIPTION OF EMBODIMENTS

The terminology used herein is for the purpose of describing particular embodiments only and is not intended to be limiting.

Before describing several exemplary embodiments, it is to be understood that the embodiments is not limited to the details of construction or process steps set forth in the following description. The embodiments described herein are capable of modifications and of being practiced or being carried out in various ways.

Reference throughout the present disclosure to “some embodiments,” or derivations thereof, means that a particular feature, structure, material, or characteristic described in connection with the embodiment is included in at least one embodiment. Thus, the appearances of the phrases such as “in some embodiments,” in various places throughout the present disclosure is not necessarily referring to the same embodiment, but can generally be attributed to any other embodiment. Furthermore, the particular features, structures, materials, or characteristics may be combined in any suitable manner in one or more embodiments.

As used herein, the terms “allergy associated immunoglobulin” and “AAI” refer to immunoglobulins in sera that mediate hypersensitivity to peanut allergens. These include one or more of IgE, IgD, IgA, IgM, and IgG (including IgG4).

As used herein, the terms “reactive”, “reactivity”, “recognize” and the like refer to the ability of an allergy associated immunoglobulin to bind to an allergenic epitope containing peptide. The level of reactivity indicates the concentration of AAI in the serum or plasma, with high reactivity associated with higher AAI concentrations and lower reactivity associated with lower AAI concentrations. The relative AAI concentration (i.e., the relative serum or plasma reactivity) is determined by the amount of signal detected in the assay. The level of reactivity of AAI to allergenic epitope containing peptides also indicates the intensity of the allergic response (i.e., higher reactivity is associated with a more intense allergic reaction).

As used herein, the term “clinical tolerance” refers to immunological tolerance to a peanut allergen that is developed by an allergic subject as a result of exposure to the allergen (i.e., tolerance developed as a result of immunotherapy).

As used herein, the term “natural tolerance” refers to immunological tolerance to a peanut allergen that is developed by an allergic subject as a biochemical process over time, either as a result of natural exposure to the allergen during a lifetime or in the absence of exposure.

The present disclosure provides peanut peptide compositions. In some embodiments, the composition consists of two peanut peptides. In some embodiments, both peanut peptides are derived from the ara h 2 peanut allergen.

In some embodiments, the first peanut peptide in the composition comprises the amino acid sequence WELQGDRRCQSQLER (ara h2 2.008; SEQ ID NO:1). In some embodiments, the first peanut peptide in the composition comprises the amino acid sequence according to SEQ ID NO:1, but having one to four conservative amino acid substitutions therein. In some embodiments, the first peanut peptide in the composition comprises the amino acid sequence according to SEQ ID NO:1, but having one conservative amino acid substitution therein. In some embodiments, the first peanut peptide in the composition comprises the amino acid sequence according to SEQ ID NO:1, but having two conservative amino acid substitutions therein. In some embodiments, the first peanut peptide in the composition comprises the amino acid sequence according to SEQ ID NO:1, but having three conservative amino acid substitutions therein. In some embodiments, the first peanut peptide in the composition comprises the amino acid sequence according to SEQ ID NO:1, but having four conservative amino acid substitutions therein.

In some embodiments, the second peanut peptide in the composition comprises the amino acid sequence DSYERDPYSPSQDPY (ara h2 2.019; SEQ ID NO:2). In some embodiments, the first peanut peptide in the composition comprises the amino acid sequence according to SEQ ID NO:2, but having one to four conservative amino acid substitutions therein. In some embodiments, the first peanut peptide in the composition comprises the amino acid sequence according to SEQ ID NO:2, but having one conservative amino acid substitution therein. In some embodiments, the first peanut peptide in the composition comprises the amino acid sequence according to SEQ ID NO:2, but having two conservative amino acid substitutions therein. In some embodiments, the first peanut peptide in the composition comprises the amino acid sequence according to SEQ ID NO:2, but having three conservative amino acid substitutions therein. In some embodiments, the first peanut peptide in the composition comprises the amino acid sequence according to SEQ ID NO:2, but having four conservative amino acid substitutions therein.

Conservative amino acid substitutions are most often classified on the basis of the amino acid structure and the general chemical characteristics of their side chains (R groups). For example, aliphatic amino acids include glycine, alanine, valine, leucine, and isoleucine, and each of these amino acids can be substituted for one another. Hydroxyl or sulfur/selenium-containing amino acids include serine, cysteine, selenocysteine, threonine, and methionine, and each of these amino acids can be substituted for one another. Aromatic amino acids include phenylalanine, tyrosine, and tryptophan, and each of these amino acids can be substituted for one another. Basic amino acids include histidine, lysine, and arginine, and each of these amino acids can be substituted for one another. Acidic or amide-containing amino acids include aspartate, glutamate, asparagine, and glutamine, and each of these amino acids can be substituted for one another.

In some embodiments, each peanut peptide is coupled to a solid support. In some embodiments, the solid support is a microsphere bead, a glass array, a silicone array, a membrane, or a microtiter plate. In some embodiments, the solid support is a glass array. In some embodiments, the solid support is a silicone array. In some embodiments, the solid support is a membrane. In some embodiments, the solid support is a microtiter plate. In some embodiments, the solid support is a microsphere bead. In some embodiments, the microsphere bead is an avidin-coupled microsphere bead. In some embodiments, the bead is a Luminex bead such as Mag™ Avidin bead or LumAvidin® bead. In some embodiments, each of the solid supports is coupled to a single peanut peptide.

In some embodiments, each of the peanut peptides is coupled to the solid support by a linker-spacer. In some embodiments, the linker-spacer comprises a linker chosen from biotin, a thiol, a hydrazine, and an amine. In some embodiments, the linker is biotin. In some embodiments, the linker is a thiol. In some embodiments, the linker is a hydrazine. In some embodiments, the linker is an amine. In some embodiments, the linker-spacer comprises a spacer chosen from a polypeptide, an oligonucleotide, an alkyl group, and a polyethylene glycol (PEG) group. In some embodiments, the spacer is a polypeptide. In some embodiments, the spacer is an oligonucleotide. In some embodiments, the spacer is an alkyl group. In some embodiments, the alkyl group is a C₁-C₁₈alkyl group or a C₃-C₁₂alkyl group. In some embodiments, the spacer is a PEG group. In some embodiments, the PEG group is PEG1 to PEG18. In some embodiments, the PEG group is PEG12. In some embodiments, the spacer is an alkyl group or a PEG group. In some embodiments, the C-terminus of each of the peanut peptides is coupled to the solid support by the linker-spacer. In some embodiments, the N-terminus of each of the peanut peptides is coupled to the solid support by the linker-spacer. In some embodiments, the C-terminus of each of the peanut peptides is coupled to the solid support by a biotin-PEG12 linker-spacer.

In some embodiments, the peanut peptides can be coupled to the solid support, which each component having one half of a pair of click chemistry linkers. For example, one of the peanut peptides and solid support can have one click chemistry linker, while the other of the peanut peptides and solid support can have a corresponding click chemistry linker. Examples of click chemistry linker pairs include, but are not limited to, azide-DBCO, amine-NHS ester, and thiol-malamide.

The present disclosure also provides methods for diagnosing a peanut allergy in a subject. The methods comprise contacting two peanut peptides coupled to a solid support with a biological sample obtained from the subject. The contacting occurs under conditions sufficient to permit binding of one or more allergy associated immunoglobulins (AAIs) in the biological sample to the two peanut peptides to form AAI-peptide-solid support complexes. The methods also comprise contacting the AAI-peptide-solid support complexes with an AAI-specific labeling reagent to form labeling reagent-AAI-peptide-solid support complexes. The methods also comprise measuring the binding of the AAI-specific labeling reagent to each AAI-peptide-solid support complex. When the combined binding for each peanut peptide in the AAI-peptide solid support complex to the AAI-specific labeling reagent is greater than a threshold value, the subject is allergic to peanuts. When the combined binding for each peanut peptide in the AAI-peptide solid support complex to the AAI-specific labeling reagent is equal to or less than the threshold value, the subject is not allergic to peanuts. The steps described herein comprise an assay for detecting the presence of specific AAIs in the biological sample to the two peanut peptides.

Any of the two peanut peptides coupled to any of the solid supports described herein can be used. For example, in some embodiments, one of the two peanut peptides comprises the amino acid sequence according to SEQ ID NO:1, or an amino acid sequence comprising SEQ ID NO:1 but having one to four conservative amino acid substitutions therein, and the other of the two peanut peptides comprises the amino acid sequence according to SEQ ID NO:2, or an amino acid sequence comprising SEQ ID NO:2 but having one to four conservative amino acid substitutions therein. In some embodiments, one of the two peanut peptides comprises the amino acid sequence according to SEQ ID NO:1, and the other of the two peanut peptides comprises the amino acid sequence according to SEQ ID NO:2. In some embodiments, these peanut peptides are coupled to a microsphere bead. Each of the peanut peptides can be coupled to the solid support by any of the linker-spacers described herein. Each of the peanut peptides can be coupled to the solid support by their C-terminal or N-terminal ends as described herein.

The biological sample can be any biological sample obtained from a subject. In some embodiments, the biological sample is chosen from serum, plasma, saliva, or a buccal swab. In some embodiments, the biological sample is serum or plasma. In some embodiments, the biological sample is serum. In some embodiments, the biological sample is plasma. In some embodiments, the biological sample is saliva. In some embodiments, the biological sample is a buccal swab.

The AAIs that may be present in the biological sample from a subject may include any one or more of IgM, IgA, IgD, IgG, and/or IgE. In some embodiments, the AAI in the biological sample is IgM, IgA, and/or IgD. In some embodiments, the AAI in the biological sample is IgG and/or IgE. In some embodiments, the AAI in the biological sample is IgE.

In some embodiments, the AAI-specific labeling reagent is a detectably labeled anti-human antibody. In some embodiments, the detectably labeled anti-human antibody is detectably labeled anti-human IgA antibody. In some embodiments, the detectably labeled anti-human antibody is detectably labeled anti-human IgD antibody. In some embodiments, the detectably labeled anti-human antibody is detectably labeled anti-human IgM antibody. In some embodiments, the detectably labeled anti-human antibody is detectably labeled anti-human IgG antibody. In some embodiments, the detectably labeled anti-human antibody is detectably labeled anti-human IgE antibody.

In some embodiments, the detectable label of the AAI-specific labeling reagent is chosen from phycoerythrin (PE), a cyanine dye, a fluorescent dye, an infrared dye, a chromogenic dye, an enzyme label, and a radioactive label. In some embodiments, the detectable label of the AAI-specific labeling reagent is PE. In some embodiments, the detectable label of the AAI-specific labeling reagent is a cyanine dye. In some embodiments, the cyanine dye is Cy3 or Cy5. In some embodiments, the detectable label of the AAI-specific labeling reagent is a fluorescent dye. In some embodiments, the fluorescent dye is Texas Red or Alexa-fluor. In some embodiments, the detectable label of the AAI-specific labeling reagent is an infrared (IR) dye. In some embodiments, the detectable label of the AAI-specific labeling reagent is a chromogenic dye. In some embodiments, the detectable label of the AAI-specific labeling reagent is an enzyme label. In some embodiments, the detectable label of the AAI-specific labeling reagent is a radioactive label. In some embodiments, the enzyme label is horse radish peroxidase (HRP) or alkaline phosphatase. In some embodiments, the detectable label of the AM-specific labeling reagent is HRP. In some embodiments, the detectable label of the AAI-specific labeling reagent is alkaline phosphatase. In some embodiments, the AAI-specific labeling reagent is a PE-labeled anti-human IgE antibody. In some embodiments, a single detectable label can generally be used for universal detection of all complexes.

In some embodiments, the anti-human AAI antibody may be conjugated to a reporter moiety that is not directly detectable, so specific binding of a second, directly detectable reporter moiety to the labeling reagent is necessary for analysis of binding. For example, a biotin-conjugated anti-AAI antibody can be used in combination with a streptavidin-conjugated fluorescent dye for detection of the biotin-conjugated anti-AAI. Examples of indirectly-detectable reporter moieties include biotin, digoxigenin, and other haptens that are detectable upon subsequent binding of a secondary antibody (e.g., anti-digoxigenin) or other binding partner (e.g., streptavidin) which is labeled for direct detection.

In some embodiments, the measuring of the binding of the AAI-specific labeling reagent to each AAI-peptide-solid support complex is carried out by a point of care device. In some embodiments, the point of care device is a multiplex peptide-bead flow cytometric analysis device or a lateral flow assay device. In some embodiments, the detectable label can be observed via silver staining, quantum dots, or refraction methodologies.

Any of the foregoing embodiments may be in the form of a microarray immunoassay, wherein each of the two peanut peptides is bound to a separate well of a microtiter plate and reacted with a biological sample to bind AAI. The peanut peptides may also be used in a lateral flow immunoassay format, wherein each peptide is immobilized in a discrete area on a porous or chromatographic support, and the serum or plasma is wicked through the support to contact the peptides for binding of AAI to the peptides. In this assay, the AAI-specific labeling reagent may comprise a chromophore or dye conjugated to anti-AAI antibody. The labeling reagent is also wicked through the support to contact the peptide-AAI complexes for binding of the labeling reagent to the complex, which indicates the presence or absence in the serum or plasma of an antibody to the peanut peptide immobilized at each discrete location of the support.

Any of the foregoing embodiments may also be in the form of a flow cytometry assay in which each peanut peptide is coupled to a separately identifiable solid support suitable for analysis by flow cytometry, such as a bead. In some embodiments, the bead with the coupled peptide is contacted with the biological sample of a subject to bind any peptide-specific AAI that is bound to the bead via the peptide, thus forming a peptide-AAI complex on the bead. An AAI-specific labeling reagent comprising, for example, a fluorescent reporter moiety, is then bound to the peptide-AAI complexes and the beads are analyzed quantitatively or qualitatively by flow cytometry. This detects fluorescence from the bound labeling reagent associated with each bead to which the peanut peptide is coupled.

In some embodiments, the flow cytometry assay may be a multiplex assay, such as provided by Luminex, which uses a microsphere array platform for quantitation and detection of peptides and proteins. Each of the peanut peptides is bound to a set of beads with the same or different spectral properties which can be used to quantify the associated peanut peptide bound to AAI by flow cytometry. The sets of beads are then contacted with the biological sample of a subject to bind peptide-recognizing AAI to each bead to form a peptide-AAI complex on the bead, and an AAI-specific labeling reagent comprising, for example, a fluorescent reporter moiety bound to the AAI of the complex. The beads are analyzed by monitoring the spectral properties of each bead and the amount of associated fluorescence from the bound labeling reagent. This process allows quantification of the peptide on the bead, and the presence or absence of AAI that is reactive to it. Results of the assay are interpreted as discussed herein.

A particularly useful quantitative assay for use in any of the methods described herein is a multiplex peptide-bead assay for flow cytometric analysis, such as the LUMINEX exMAP multiplex bead assay, which is a high-throughput alternative to the ELISA. In this assay, polystyrene beads (microspheres) dyed with distinct proportions of red and near-infrared fluorophores are used as the solid support. The peptides may be chemically linked to the beads or bound thereto through peptide-specific capture antibodies coated on the beads. The proportions of the fluorophores define a “spectral address” for each bead population that can be identified by a flow cytometer using digital signal processing. Detection of a third fluorescence color is used for measurement of the fluorescence intensity of the reporter moiety of the labeling reagent bound to the bead. Multiple analytes can be detected simultaneously by binding each peptide to a bead having a specific “spectral address.” Contacting the beads with a biological sample containing AAI that are specific for the peptide bound to it is followed by addition of anti-human AAI antibodies conjugated to a reporter moiety. In some embodiments, the reporter moiety of the anti-human AAI is biotin and binding to phycoerythyrin (PE)-conjugated streptavidin provides the fluorescent signal for detection. Following binding of the labeling reagent, the beads are analyzed on a dual-laser flow-based detection instrument, such as the LUMINEX 200 or Bio-Rad BIO-PLEX analyzer. One laser classifies the bead and identifies the peptide bound to it. The second laser determines the magnitude of the reporter-derived signal, which is in direct proportion to the amount of bound serum or plasma AAI.

In some embodiments, measuring the binding of the AAI-specific labeling reagent to each AAI-peptide-solid support complex comprises measuring the mean fluorescent intensity (MFI) of each AAI-specific labeling reagent. When the combined MFI binding for each peanut peptide in the AAI-peptide solid support complex to the AAI-specific labeling reagent is greater than a threshold value, the subject is allergic to peanuts. When the combined MFI for each peanut peptide in the AAI-peptide solid support complex to the AAI-specific labeling reagent is equal to or less than the threshold value, the subject is not allergic to peanuts. In some embodiments, when h2.008+(h2.019)/20 is <0.20, the subject is not allergic to peanuts. In some embodiments, when h2.008+(h2.019)/20 is >0.20, the subject is allergic to peanuts. In these equations, h2.008 is the calibrated net MFI of the labeling reagent-AAI-peptide-solid support complex wherein the peanut peptide comprises SEQ ID NO:1, and h2.019 is the calibrated net MFI of the labeling reagent-AAI-peptide-solid support complex wherein the peanut peptide comprises SEQ ID NO:2. In some embodiments, the AAI-specific labeling reagent is any of the detectably labeled anti-human antibodies described herein. In some embodiments, the MFI of each AAI-specific labeling reagent is background subtracted.

In some embodiments, the methods further comprise performing or having performed a Skin Prick Test (SPT) and/or a total peanut specific IgE (sIgE) test. In some embodiments, the methods further comprise performing or having performed a SPT. In some embodiments, the methods further comprise performing or having performed an sIgE test. In some embodiments, when the SPT is <3 mm and/or the sIgE is <0.10 kU/L then the subject is not allergic to peanuts, and when the SPT is >18 mm and/or the sIgE is >18 kU/L then the subject is allergic to peanuts.

The present disclosure also provides methods for detecting development of clinical tolerance to peanuts in a subject that is allergic to peanuts. The methods comprise contacting two peanut peptides coupled to a solid support with a biological sample obtained from the subject under conditions sufficient to permit binding of one or more allergy associated immunoglobulins (AAIs) in the biological sample to the two peanut peptides to form two AAI-peptide-solid support complexes. The AAI-peptide-solid support complexes are then contacted with an AAI-specific labeling reagent to form labeling reagent-AAI-peptide-solid support complexes. The methods comprise measuring the binding of the AAI-specific labeling reagent to each AAI-peptide-solid support complex. The methods also comprise comparing the binding of the AAI-specific labeling reagent to each AAI-peptide-solid support complex to the binding of the AAI-specific labeling reagent to each AAI-peptide-solid support complex from a biological sample previously obtained from the subject. When the combined binding for each peanut peptide in the AAI-peptide solid support complex to the AAI-specific labeling reagent is greater than or equal to the combined binding for each peanut peptide in the AAI-peptide solid support complex to the AAI-specific labeling reagent for the previously obtained biological sample, the subject has not established clinical tolerance to peanuts. When the combined binding for each peanut peptide in the AAI-peptide solid support complex to the AAI-specific labeling reagent is less than the combined binding for each peanut peptide in the AAI-peptide solid support complex to the AAI-specific labeling reagent for the previously obtained biological sample, the subject has established clinical tolerance to peanuts.

Any of the two peanut peptides coupled to any of the solid supports described herein can be used. For example, in some embodiments, one of the two peanut peptides comprises the amino acid sequence according to SEQ ID NO:1, or an amino acid sequence comprising SEQ ID NO:1 but having one to four conservative amino acid substitutions therein, and the other of the two peanut peptides comprises the amino acid sequence according to SEQ ID NO:2, or an amino acid sequence comprising SEQ ID NO:2 but having one to four conservative amino acid substitutions therein. In some embodiments, one of the two peanut peptides comprises the amino acid sequence according to SEQ ID NO:1, and the other of the two peanut peptides comprises the amino acid sequence according to SEQ ID NO:2. In some embodiments, these peanut peptides are coupled to a microsphere bead. Each of the peanut peptides can be coupled to the solid support by any of the linker-spacers described herein. Each of the peanut peptides can be coupled to the solid support by their C-terminal or N-terminal ends as described herein.

The biological sample can be any biological sample obtained from a subject. In some embodiments, the biological sample is chosen from serum, plasma, saliva, or a buccal swab. In some embodiments, the biological sample is serum or plasma. In some embodiments, the biological sample is serum. In some embodiments, the biological sample is plasma. In some embodiments, the biological sample is saliva. In some embodiments, the biological sample is a buccal swab.

The AAIs that may be present in the biological sample from a subject may include any one or more of IgM, IgA, IgD, IgG, and/or IgE. In some embodiments, the AAI in the biological sample is IgM, IgA, and/or IgD. In some embodiments, the AAI in the biological sample is IgG and/or IgE. In some embodiments, the AAI in the biological sample is IgE.

In some embodiments, the AAI-specific labeling reagent is a detectably labeled anti-human antibody. In some embodiments, the detectably labeled anti-human antibody is detectably labeled anti-human IgA antibody. In some embodiments, the detectably labeled anti-human antibody is detectably labeled anti-human IgD antibody. In some embodiments, the detectably labeled anti-human antibody is detectably labeled anti-human IgM antibody. In some embodiments, the detectably labeled anti-human antibody is detectably labeled anti-human IgG antibody. In some embodiments, the detectably labeled anti-human antibody is detectably labeled anti-human IgE antibody.

In some embodiments, the detectable label of the AAI-specific labeling reagent is chosen from phycoerythrin (PE), a cyanine dye, a fluorescent dye, an infrared dye, a chromogenic dye, an enzyme label, and a radioactive label. In some embodiments, the detectable label of the AAI-specific labeling reagent is PE. In some embodiments, the detectable label of the AAI-specific labeling reagent is a cyanine dye. In some embodiments, the cyanine dye is Cy3 or Cy5. In some embodiments, the detectable label of the AAI-specific labeling reagent is a fluorescent dye. In some embodiments, the fluorescent dye is Texas Red or Alexa-fluor. In some embodiments, the detectable label of the AAI-specific labeling reagent is an IR dye. In some embodiments, the detectable label of the AAI-specific labeling reagent is a chromogenic dye. In some embodiments, the detectable label of the AAI-specific labeling reagent is an enzyme label. In some embodiments, the detectable label of the AAI-specific labeling reagent is a radioactive label. In some embodiments, the enzyme label is horse radish peroxidase (HRP) or alkaline phosphatase. In some embodiments, the detectable label of the AAI-specific labeling reagent is HRP. In some embodiments, the detectable label of the AAI-specific labeling reagent is alkaline phosphatase. In some embodiments, the AAI-specific labeling reagent is a PE-labeled anti-human IgE antibody. In some embodiments, a single detectable label can generally be used for universal detection of all complexes.

In some embodiments, the anti-human AAI antibody may be conjugated to a reporter moiety that is not directly detectable, so specific binding of a second, directly detectable reporter moiety to the labeling reagent is necessary for analysis of binding. For example, a biotin-conjugated anti-AAI antibody can be used in combination with a streptavidin-conjugated fluorescent dye for detection of the biotin-conjugated anti-AAI. Examples of indirectly-detectable reporter moieties include biotin, digoxigenin, and other haptens that are detectable upon subsequent binding of a secondary antibody (e.g., anti-digoxigenin) or other binding partner (e.g., streptavidin) which is labeled for direct detection.

In some embodiments, the measuring of the binding of the AAI-specific labeling reagent to each AAI-peptide-solid support complex is carried out by a point of care device. In some embodiments, the point of care device is a multiplex peptide-bead flow cytometric analysis device or a lateral flow assay device. In some embodiments, the detectable label can be observed via silver staining, quantum dots, or refraction methodologies.

Any of the foregoing embodiments may be in the form of a microarray immunoassay, wherein each of the two peanut peptides is bound to a separate well of a microtiter plate and reacted with a biological sample to bind AAI. The peanut peptides may also be used in a lateral flow immunoassay format, wherein each peptide is immobilized in a discrete area on a porous or chromatographic support, and the serum or plasma is wicked through the support to contact the peptides for binding of AAI to the peptides. In this assay, the AAI-specific labeling reagent may comprise a chromophore or dye conjugated to anti-AAI antibody. The labeling reagent is also wicked through the support to contact the peptide-AAI complexes for binding of the labeling reagent to the complex, which indicates the presence or absence in the serum or plasma of an antibody to the peanut peptide immobilized at each discrete location of the support.

Any of the foregoing embodiments may also be in the form of a flow cytometry assay in which each peanut peptide is coupled to a separately identifiable solid support suitable for analysis by flow cytometry, such as a bead. In some embodiments, the bead with the coupled peptide is contacted with the biological sample of a subject to bind any peptide-specific AAI that is bound to the bead via the peptide, thus forming a peptide-AAI complex on the bead. An AAI-specific labeling reagent comprising, for example, a fluorescent reporter moiety, is then bound to the peptide-AAI complexes and the beads are analyzed quantitatively or qualitatively by flow cytometry. This detects fluorescence from the bound labeling reagent associated with each bead to which the peanut peptide is coupled.

In some embodiments, the flow cytometry assay may be a multiplex assay, such as provided by Luminex, which uses a microsphere array platform for quantitation and detection of peptides and proteins. Each of the peanut peptides is bound to a set of beads with the same or different spectral properties which can be used to quantify the associated peanut peptide bound to AAI by flow cytometry. The sets of beads are then contacted with the biological sample of a subject to bind peptide-recognizing AAI to each bead to form a peptide-AAI complex on the bead, and an AAI-specific labeling reagent comprising, for example, a fluorescent reporter moiety bound to the AAI of the complex. The beads are analyzed by monitoring the spectral properties of each bead and the amount of associated fluorescence from the bound labeling reagent. This process allows quantification of the peptide on the bead, and the presence or absence of AAI that is reactive to it. Results of the assay are interpreted as discussed herein.

A particularly useful quantitative assay for use in any of the methods described herein is a multiplex peptide-bead assay for flow cytometric analysis, such as the LUMINEX exMAP multiplex bead assay, which is a high-throughput alternative to the ELISA. In this assay, polystyrene beads (microspheres) dyed with distinct proportions of red and near-infrared fluorophores are used as the solid support. The peptides may be chemically linked to the beads or bound thereto through peptide-specific capture antibodies coated on the beads. The proportions of the fluorophores define a “spectral address” for each bead population that can be identified by a flow cytometer using digital signal processing. Detection of a third fluorescence color is used for measurement of the fluorescence intensity of the reporter moiety of the labeling reagent bound to the bead. Multiple analytes can be detected simultaneously by binding each peptide to a bead having a specific “spectral address.” Contacting the beads with a biological sample containing AAI that are specific for the peptide bound to it is followed by addition of anti-human AAI antibodies conjugated to a reporter moiety. In some embodiments, the reporter moiety of the anti-human AAI is biotin and binding to phycoerythyrin (PE)-conjugated streptavidin provides the fluorescent signal for detection. Following binding of the labeling reagent, the beads are analyzed on a dual-laser flow-based detection instrument, such as the LUMINEX 200 or Bio-Rad BIO-PLEX analyzer. One laser classifies the bead and identifies the peptide bound to it. The second laser determines the magnitude of the reporter-derived signal, which is in direct proportion to the amount of bound serum or plasma AAI.

In some embodiments, measuring the binding of the AAI-specific labeling reagent to each AAI-peptide-solid support complex comprises measuring the MFI of each AAI-specific labeling reagent as described herein. The methods also comprise comparing the binding of the AAI-specific labeling reagent to each AAI-peptide-solid support complex to the binding of the AAI-specific labeling reagent to each AAI-peptide-solid support complex from a biological sample previously obtained from the subject. When the combined binding for each peanut peptide in the AAI-peptide solid support complex to the AAI-specific labeling reagent is greater than or equal to the combined binding for each peanut peptide in the AAI-peptide solid support complex to the AAI-specific labeling reagent for the previously obtained biological sample, the subject has not established clinical tolerance to peanuts. When the combined binding for each peanut peptide in the AAI-peptide solid support complex to the AAI-specific labeling reagent is less than the combined binding for each peanut peptide in the AAI-peptide solid support complex to the AAI-specific labeling reagent for the previously obtained biological sample, the subject has established clinical tolerance to peanuts. In some embodiments, the MFI of each AAI-specific labeling reagent is background subtracted.

The age of a subject undergoing examination for development of clinical tolerance can be from about 2 years old to 17 years old. In some embodiments, the subject is less than about one year old, less than about 2 years old, less than about 3 years old, less than about 4 years old, less than about 5 years old, or less than about 6 years old.

In some embodiments, the initial detection of development of clinical tolerance can be used to predict if a subject will either develop a natural tolerance to the allergy or be responsive to therapy. In some embodiments, an allergic subject is exposed to the immunogen (immunotherapy) prior to analyzing the initial profile. If at the subsequent time-point there is a reduction of at least 2-fold in serum concentration of all AAIs to the two peanut peptides described herein in the initial profile, it is likely that the subject will develop either clinical or natural tolerance to peanuts.

The present disclosure also provides methods of desensitizing an infant to two peanut allergens to induce tolerance or non-allergy to peanuts. In some embodiments, the methods comprise administering two peanut peptides to the infant. One of the two peanut peptides comprises the amino acid sequence WELQGDRRCQSQLER (SEQ ID NO:1) or an amino acid sequence comprising SEQ ID NO:1 but having one to four conservative amino acid substitutions therein. The other of the two peanut peptides comprises the amino acid sequence DSYERDPYSPSQDPY (SEQ ID NO:2) or an amino acid sequence comprising SEQ ID NO:2 but having one to four conservative amino acid substitutions therein. Any of the peanut peptides described herein can be used. In some embodiments, one of the two peanut peptides comprises the amino acid sequence according to SEQ ID NO:1, and the other of the two peanut peptides comprises the amino acid sequence according to SEQ ID NO:2. These peptides may have certain utility for the desensitization of peanut allergy either individually, in combination, or in combination with other therapeutic approaches.

The peanut peptides can be administered via an oral, sublingual, intradermal, sub-cutaneous, inhaled, or epicutaneous route to induce desensitization. The age of a subject undergoing desensitization can be less than about one year old, less than about 2 years old, less than about 3 years old, less than about 4 years old, less than about 5 years old, or less than about 6 years old. The amount of total peptide or individual peptide can be about 1 gram or less per dose.

The present disclosure also provides kits for carrying out any of the methods described herein. In some embodiments, the kit comprises a solid support coupled to a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein. The kits also comprise an allergy associated immunoglobulin (AAI)-specific labeling reagent.

The kits described herein may also comprise additional components. In some embodiments, the kit further comprises instructions for use. In some embodiments, the kit further comprises one or more of a binding buffer, a wash buffer, a detection buffer, a non-allergic control sample, a negative buffer control sample, and an allergic positive control sample. In some embodiments, peptides containing non-reactive epitopes of peanut proteins can be used as negative controls.

The peptides coupled to the solid support can be any of the peanut peptides described herein and can be coupled to the solid support by any of the means described herein. The solid supports can be any of the solid supports described herein. The AAI-specific labeling reagents can be any of the AAI-specific labeling reagents described herein. The detectable label for any of the AAI-specific labeling reagents can be any of the detectable labels described herein.

The subject matter described herein has many advantages. First, the methods identify allergic and non-allergic subjects with confidence (e.g., PPV=95% and NPV=92%). Second, the methods may allow medical practitioners to de-list existing patients, possibly up to two-thirds, with indeterminate test results and/or ambiguous clinical history, significantly reduce overdiagnosis by minimizing false positive, and improve quality of life issues (e.g., anxiety, bullying, need for peanut-free environments). Third, the methods provide a better way to monitor a subject's disease status over time, to monitor allergy outgrowth over time, and to provide a quantitative measure of disease status over time. Fourth, the methods can be used to provide a better treatment guidance. For example, the methods can be used to enhance the decision for or against oral food challenge (OFC) or therapy, optimize the use of OFC, and identify appropriate candidates for therapy with confidence. Fifth, the methods described herein reduce costs. For example, the methods can reduce the need for additional testing (e.g., OFC, sIgE, and component-resolved diagnosis (CRD), eliminate costs associated with food allergy by de-labeling those misdiagnosed, eliminate costs associated with unnecessary immunotherapy by de-labeling, de-labeled subjects will no longer need EpiPens purchasing and restocking, noon further need to purchase non-peanut foods, and a reduction in other costs such as, for example, sick-days in the work place.

The following representative embodiments are presented:

Embodiment 1. A method for diagnosing a peanut allergy in a subject comprising: contacting two peanut peptides coupled to a solid support with a biological sample obtained from the subject under conditions sufficient to permit binding of one or more allergy associated immunoglobulins (AAIs) in the biological sample to the two peanut peptides to form AAI-peptide-solid support complexes, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein; contacting the AAI-peptide-solid support complexes with an AAI-specific labeling reagent to form labeling reagent-AAI-peptide-solid support complexes; and measuring the binding of the AAI-specific labeling reagent to each AAI-peptide-solid support complex; wherein when the combined binding for each peanut peptide in the AAI-peptide solid support complex to the AAI-specific labeling reagent is greater than a threshold value, the subject is allergic to peanuts, and when the combined binding for each peanut peptide in the AAI-peptide solid support complex to the AAI-specific labeling reagent is equal to or less than the threshold value, the subject is not allergic to peanuts.

Embodiment 2. The method according to embodiment 1, wherein the biological sample is chosen from serum, plasma, saliva, or a buccal swab.

Embodiment 3. The method according to embodiment 1, wherein the biological sample is serum or plasma.

Embodiment 4. The method according to any one of embodiments 1 to 3, wherein one of the two peanut peptides comprises the amino acid sequence according to SEQ ID NO:1, and the other of the two peanut peptides comprises the amino acid sequence according to SEQ ID NO:2.

Embodiment 5. The method according to any one of embodiments 1 to 4, wherein each of the peanut peptides is coupled to the solid support by a linker-spacer.

Embodiment 6. The method according to embodiment 5, wherein the linker-spacer comprises a linker chosen from biotin, thiol, hydrazine, and amine, and a spacer chosen from a polypeptide, an oligonucleotide, an alkyl group, and a polyethylene glycol (PEG) group. Embodiment 7. The method according to embodiment 6, wherein the spacer is an alkyl group or a PEG group.

Embodiment 8. The method according to embodiment 7, wherein the alkyl group is a C₁-C₁₈alkyl group.

Embodiment 9. The method according to embodiment 7, wherein the PEG group is PEG1 to PEG18.

Embodiment 10. The method according to embodiment 9, wherein the PEG group is PEG-12.

Embodiment 11. The method according to any one of embodiments 5 to 10, wherein the C-terminus of each of the peanut peptides is coupled to the solid support by the linker-spacer.

Embodiment 12. The method according to any one of embodiments 5 to 10, wherein the N-terminus of each of the peanut peptides is coupled to the solid support by the linker-spacer.

Embodiment 13. The method according to embodiment 5, wherein the C-terminus of each of the peanut peptides is coupled to the solid support by a biotin-PEG12 linker-spacer.

Embodiment 14. The method according to any one of embodiments 1 to 13, wherein the solid support is a microsphere bead, glass array, silicone array, membrane, or microtiter plate.

Embodiment 15. The method according to embodiment 14, wherein the solid support is a microsphere bead.

Embodiment 16. The method according to embodiment 15, wherein the microsphere bead is an avidin-coupled microsphere bead.

Embodiment 17. The method according to any one of embodiments 1 to 16, wherein the AAI is IgM, IgA, and/or IgD.

Embodiment 18. The method according to any one of embodiments 1 to 16, wherein the AAI is IgG and/or IgE.

Embodiment 19. The method according to any one of embodiments 1 to 16, wherein the AAI is IgE.

Embodiment 20. The method according to any one of embodiments 1 to 19, wherein the AAI-specific labeling reagent is a detectably labeled anti-human antibody.

Embodiment 21. The method according to embodiment 20, wherein the detectably labeled anti-human antibody is detectably labeled anti-human IgE antibody.

Embodiment 22. The method according to embodiment 20 or embodiment 21, wherein the detectable label of the AAI-specific labeling reagent is chosen from phycoerythrin (PE), a cyanine dye, a fluorescent dye, an infrared dye, a chromogenic dye, an enzyme label, and a radioactive label.

Embodiment 23. The method according to embodiment 22, wherein the detectable label is PE.

Embodiment 24. The method according to embodiment 20, wherein the AAI-specific labeling reagent is a PE-labeled anti-human IgE antibody.

Embodiment 25. The method according to any one of embodiments 1 to 24, wherein the measuring of the binding of the AAI-specific labeling reagent to each AAI-peptide-solid support complex is carried out by a point of care device.

Embodiment 26. The method according to embodiment 25, wherein the point of care device is a multiplex peptide-bead flow cytometric analysis device or a lateral flow assay device.

Embodiment 27. The method according to any one of embodiments 1 to 26, wherein measuring the binding of the AAI-specific labeling reagent to each AAI-peptide-solid support complex comprises measuring the mean fluorescent intensity (MFI) of each AAI-specific labeling reagent, wherein: when h2.008+(h2.019)/20 is <0.20, the subject is not allergic to peanuts; and when h2.008+(h2.019)/20 is >0.20, the subject is allergic to peanuts; wherein h2.008 is the calibrated net MFI of the labeling reagent-AAI-peptide-solid support complex wherein the peanut peptide comprises SEQ ID NO:1, and h2.019 is the calibrated net MFI of the labeling reagent-AAI-peptide-solid support complex wherein the peanut peptide comprises SEQ ID NO:2; and wherein the AM-specific labeling reagent is a detectably labeled anti-human antibody.

Embodiment 28. The method according to embodiment 27, wherein the MFI of each AAI-specific labeling reagent is background subtracted.

Embodiment 29. The method according to any one of embodiments 1 to 28, further comprising performing or having performed a skin prick test (SPT) and/or a total peanut specific IgE (sIgE) test.

Embodiment 30. The method according to embodiment 29, wherein when the SPT is ≤3 mm and/or the sIgE is ≤0.10 kU/L then the subject is not allergic to peanuts, and when the SPT is ≥18 mm and/or the sIgE is ≥18 kU/L then the subject is allergic to peanuts.

Embodiment 31. A method for detecting development of clinical tolerance to peanuts in a subject that is allergic to peanuts comprising: contacting two peanut peptides coupled to a solid support with a biological sample obtained from the subject under conditions sufficient to permit binding of one or more allergy associated immunoglobulins (AAIs) in the biological sample to the two peanut peptides to form two AAI-peptide-solid support complexes, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein; contacting the AAI-peptide-solid support complexes with an AAI-specific labeling reagent to form labeling reagent-AAI-peptide-solid support complexes; measuring the binding of the AAI-specific labeling reagent to each AAI-peptide-solid support complex; and comparing the binding of the AAI-specific labeling reagent to each AAI-peptide-solid support complex to the binding of the AAI-specific labeling reagent to each AAI-peptide-solid support complex from a biological sample previously obtained from the subject; wherein when the combined binding for each peanut peptide in the AAI-peptide solid support complex to the AAI-specific labeling reagent is greater than or equal to the combined binding for each peanut peptide in the AAI-peptide solid support complex to the AAI-specific labeling reagent for the previously obtained biological sample, the subject has not established clinical tolerance to peanuts; and when the combined binding for each peanut peptide in the AAI-peptide solid support complex to the AAI-specific labeling reagent is less than the combined binding for each peanut peptide in the AAI-peptide solid support complex to the AAI-specific labeling reagent for the previously obtained biological sample, the subject has established clinical tolerance to peanuts.

Embodiment 32. The method according to embodiment 31, wherein the biological sample is chosen from serum, plasma, saliva, or a buccal swab.

Embodiment 33. The method according to embodiment 31, wherein the biological sample is serum or plasma.

Embodiment 34. The method according to any one of embodiments 31 to 33, wherein one of the two peanut peptides comprises the amino acid sequence according to SEQ ID NO:1, and the other of the two peanut peptides comprises the amino acid sequence according to SEQ ID NO:2.

Embodiment 35. The method according to any one of embodiments 31 to 34, wherein each of the peanut peptides is coupled to the solid support by a linker-spacer.

Embodiment 36. The method according to embodiment 35, wherein the linker-spacer comprises a linker chosen from biotin, thiol, hydrazine, and amine, and a spacer chosen from a polypeptide, an oligonucleotide, an alkyl group, and a polyethylene glycol (PEG) group.

Embodiment 37. The method according to embodiment 36, wherein the spacer is an alkyl group or a PEG group.

Embodiment 38. The method according to embodiment 37, wherein the alkyl group is a C₁-C₁₈alkyl group.

Embodiment 39, The method according to embodiment 37 wherein the PEG group is PEG1 to PEG18.

Embodiment 40. The method according to embodiment 39, wherein the PEG group is PEG12.

Embodiment 41. The method according to any one of embodiments 35 to 40, wherein the C-terminus of each of the peanut peptides is coupled to the solid support by the linker-spacer.

Embodiment 42. The method according to any one of embodiments 35 to 40, wherein the N-terminus of each of the peanut peptides is coupled to the solid support by the linker-spacer.

Embodiment 43. The method according to embodiment 35, wherein the C-terminus of each of the peanut peptides is coupled to the solid support by a biotin-PEG12 linker-spacer.

Embodiment 44. The method according to any one of embodiments 31 to 43, wherein the solid support is a microsphere bead, glass array, silicone array, membrane, or microtiter plate.

Embodiment 45. The method according to embodiment 44, wherein the solid support is a microsphere bead.

Embodiment 46. The method according to embodiment 45, wherein the microsphere bead is an avidin-coupled microsphere bead.

Embodiment 47. The method according to any one of embodiments 31 to 46, wherein the AM is IgM, IgA, and/or IgD.

Embodiment 48. The method according to any one of embodiments 31 to 46, wherein the AM is IgG and/or IgE.

Embodiment 49. The method according to any one of embodiments 31 to 46, wherein the AM is IgE.

Embodiment 50. The method according to any one of embodiments 31 to 49, wherein the AM-specific labeling reagent is a detectably labeled anti-human antibody.

Embodiment 51. The method according to embodiment 50, wherein the detectably labeled anti-human antibody is detectably labeled anti-human IgE antibody.

Embodiment 52. The method according to embodiment 50 or embodiment 51, wherein the detectable label of the AAI-specific labeling reagent is chosen from phycoerythrin (PE), a cyanine dye, a fluorescent dye, an infrared dye, a chromogenic dye, an enzyme label, and a radioactive label.

Embodiment 53. The method according to embodiment 52, wherein the detectable label is PE.

Embodiment 54. The method according to embodiment 50, wherein the AAI-specific labeling reagent is a PE-labeled anti-human IgE antibody.

Embodiment 55. The method according to any one of embodiments 31 to 54, wherein the measuring of the binding of the AAI-specific labeling reagent to each AAI-peptide-solid support complex is carried out by a point of care device.

Embodiment 56. The method according to embodiment 55, wherein the point of care device is a multiplex peptide-bead flow cytometric analysis device or a lateral flow assay device.

Embodiment 57. The method according to any one of embodiments 31 to 56, wherein measuring the binding of the AAI-specific labeling reagent to each AAI-peptide-solid support complex comprises measuring the mean fluorescent intensity (MFI) of each AAI-specific labeling reagent, wherein: when h2.008+(h2.019)/20 is ≤0.20, the subject is not allergic to peanuts; and when h2.008+(h2.019)/20 is >0.20, the subject is allergic to peanuts; wherein h2.008 is the calibrated net MFI of the labeling reagent-AAI-peptide-solid support complex wherein the peanut peptide comprises SEQ ID NO:1, and h2.019 is the calibrated net MFI of the labeling reagent-AAI-peptide-solid support complex wherein the peanut peptide comprises SEQ ID NO:2; and wherein the AM-specific labeling reagent is a detectably labeled anti-human antibody.

Embodiment 58. The method according to embodiment 57, wherein the MFI of each AAI-specific labeling reagent is background subtracted.

Embodiment 59. A method of desensitizing an infant to two peanut allergens to induce tolerance or non-allergy to peanuts comprising administering two peanut peptides to the infant, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein.

Embodiment 60. The method according to embodiment 59, wherein one of the two peanut peptides comprises the amino acid sequence according to SEQ ID NO:1, and the other of the two peanut peptides comprises the amino acid sequence according to SEQ ID NO:2.

Embodiment 61. A composition consisting of a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein.

Embodiment 62. The composition according to embodiment 61, wherein the first peanut peptide comprises the amino acid sequence according to SEQ ID NO:1, and the second peanut peptide comprises the amino acid sequence according to SEQ ID NO:2.

Embodiment 63. The composition according to embodiment 61 or embodiment 62, wherein each peanut peptide is coupled to a solid support.

Embodiment 64. The composition according to any one of embodiments 61 to 63, wherein each of the peanut peptides is coupled to the solid support by a linker-spacer.

Embodiment 65. The composition according to embodiment 64, wherein the linker-spacer comprises a linker chosen from biotin, thiol, hydrazine, and amine, and a spacer chosen from a polypeptide, an oligonucleotide, an alkyl group, and a polyethylene glycol (PEG) group.

Embodiment 66. The composition according to embodiment 65, wherein the spacer is an alkyl group or a PEG group.

Embodiment 67. The composition according to embodiment 66, wherein the alkyl group is a C₁-C₁₈alkyl group.

Embodiment 68. The composition according to embodiment 66, wherein the PEG group is PEG1 to PEG18.

Embodiment 69. The composition according to embodiment 68, wherein the PEG group is PEG12.

Embodiment 70. The composition according to any one of embodiments 64 to 69, wherein the C-terminus of each of the peanut peptides is coupled to the solid support by the linker-spacer.

Embodiment 71. The composition according to any one of embodiments 64 to 69, wherein the N-terminus of each of the peanut peptides is coupled to the solid support by the linker-spacer.

Embodiment 72. The composition according to embodiment 64, wherein the C-terminus of each of the peanut peptides is coupled to the solid support by a biotin-PEG12 linker-spacer.

Embodiment 73. The composition according to any one of embodiments 60 to 72, wherein the solid support is a microsphere bead, glass array, silicone array, membrane, or microtiter plate.

Embodiment 74. The composition according to embodiment 73, wherein the solid support is a microsphere bead.

Embodiment 75. The composition according to embodiment 74, wherein the microsphere bead is an avidin-coupled microsphere bead.

Embodiment 76. A kit comprising: a solid support coupled to a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein; and an allergy associated immunoglobulin (AAI)-specific labeling reagent.

Embodiment 77. The kit according to embodiment 76, further comprising instructions for use.

Embodiment 78. The kit according to embodiment 76 or embodiment 77, further comprising one or more of a binding buffer, a wash buffer, a detection buffer, a non-allergic control sample, a negative buffer control sample, and an allergic positive control sample.

Embodiment 79. The kit according to any one of embodiments 76 to 78, wherein each of the peanut peptides is coupled to the solid support by a linker-spacer.

Embodiment 80. The kit according to embodiment 79, wherein the linker-spacer comprises a linker chosen from biotin, thiol, hydrazine, and amine, and a spacer chosen from a polypeptide, an oligonucleotide, an alkyl group, and a polyethylene glycol (PEG) group.

Embodiment 81. The kit according to embodiment 80, wherein the spacer is an alkyl group or a PEG group.

Embodiment 82. The kit according to embodiment 81, wherein the alkyl group is a C₁-C₁₈alkyl group.

Embodiment 83. The kit according to embodiment 81, wherein the PEG group is PEG1 to PEG18.

Embodiment 84. The kit according to embodiment 83, wherein the PEG group is PEG-12.

Embodiment 85. The kit according to any one of embodiments 79 to 84, wherein the C-terminus of each of the peanut peptides is coupled to the solid support by the linker-spacer.

Embodiment 86. The kit according to any one of embodiments 79 to 84, wherein the N-terminus of each of the peanut peptides is coupled to the solid support by the linker-spacer.

Embodiment 87. The kit according to embodiment 79, wherein the C-terminus of each of the peanut peptides is coupled to the solid support by a biotin-PEG12 linker-spacer.

Embodiment 88. The kit according to any one of embodiments 75 to 87, wherein the solid support is a microsphere bead, glass array, silicone array, membrane, or microtiter plate.

Embodiment 89. The kit according to embodiment 88, wherein the solid support is a microsphere bead.

Embodiment 90. The kit according to embodiment 89, wherein the microsphere bead is an avidin-coupled microsphere bead.

Embodiment 91. The kit according to any one of embodiments 75 to 90, wherein the AAI-specific labeling reagent is a detectably labeled anti-human antibody.

Embodiment 92. The kit according to embodiment 91, wherein the detectably labeled anti-human antibody is detectably labeled anti-human IgE antibody.

Embodiment 93. The kit according to embodiment 91 or embodiment 92, wherein the detectable label of the AAI-specific labeling reagent is chosen from phycoerythrin (PE), a cyanine dye, a fluorescent dye, an infrared dye, a chromogenic dye, an enzyme label, and a radioactive label.

Embodiment 94. The kit according to embodiment 93, wherein the detectable label is PE.

Embodiment 95. The kit according to embodiment 91, wherein the AAI-specific labeling reagent is a PE-labeled anti-human IgE antibody.

Embodiment 96. The kit according to any one of embodiments 75 to 95, further comprising a reporter moiety that specifically binds to the AM-specific labeling reagent.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Validation of Peanut Allergy Diagnostic

### Algorithm

Below is an example of an algorithm that integrates two epitopes into a classifier:

If h2.008+(h2.019)/20≤0.20 then “Not Allergic”, otherwise “Allergic”. For this algorithm, h2.008 and h2.019 are represented as calibrated net MFI and are determined using the equations listed below.

Buffer NetMFI=log₂(Median(Bᵣ₁,Bᵣ₂,Bᵣ₂)+1) Equation 1

Bᵣₙ is the NetMFI for the buffer control reported from the Luminex system for replicate n of the buffer control in a given batch.

Positive Control NetMFI=log₂(Median(Pᵣ₁,Pᵣ₂,Pᵣ₃)+1)−Buffer NetMFI Equation 2

Pᵣₙ is the NetMFI for the positive control reported from the Luminex system for replicate n of the positive control in a given batch.

NetMFI=log₂(Median(Sᵣ₁,Sᵣ₂,Sᵣ₃)+1)−Buffer NetMFI Equation 3

Sᵣₙ is the NetMFI for a sample reported from the Luminex system for replicate n of the sample in a given batch.

$$Correction Factor = \frac{constant}{Positive Control Net MFI} \qquad Equation 4$$

Constant is an epitope-specific correction constant.

$$\begin{matrix}Calibrated NetMFI = \{ \begin{matrix}\underline{Correction Factor * NetMFI} & \underline{\text{?} f NetMFI > 0} \\ 0 & \text{?} f NetMFI \leq 0\end{matrix} & Equation 5 \\ P . DIA .1 . Result = h {2.008}_{Calibrated NetMFI} + \frac{h {2.019}_{Calibrated NetMFI}}{20} \\ \text{?} \text{indicates text missing or illegible when filed} & Equation 6\end{matrix}$$

An additional SPT and/or sIgE test can also be integrated into the algorithm whereby any one of the three tests can confirm the allergenic status of the subject to peanuts.
If SPT≤3 mm or sIgE≤0.10 kU/L, then “Not Allergic”. Stop.
   If SPT≥18 mm or sIgE≥18 kU/L, then “Allergic”. Stop.
      For the SPT, the units are mm. For the sIgE, the units are kU/L.

### Methods

The test was performed on 133 subjects (31 allergic, 102 non-allergic) from the avoidance arm of a LEAP study. All diagnoses were determined by OFC at age 5 years. Plasma samples were obtained at years 2.5 and 5 for each subject. These samples were analyzed using BBEA methodology to obtain IgE and IgG4 epitope levels for each subject at year 2.5 and year 5. The IgE (IgG4) epitope levels for each subject were normalized by the median value of all IgE (IgG4) epitope measurements.

Data was analyzed at year 5 to determine the best performing IgE or IgG4 epitope for segregating allergic and non-allergic subjects. Specifically, the best performing IgE or IgG4 epitope is the one with the best AUC for classifying those subjects as allergic or non-allergic after initial triage of subjects by peanut specific IgE (sIgE) level below 0.1 kU/L. It was then confirmed that this same IgE or IgG4 epitope was also the best performing epitope at year 2.5. After identification of the best performing epitope and decision thresholds, the diagnostic test was completed prior to validation.

In brief, peanut peptides (CS Bio, Menlo Park, Calif., USA) were coupled to LumAvidin beads (Luminex Corporation, Austin, Tex.) and stored in PBS-TBN buffer (lx PBS+0.02% Tween20+0.1% BSA). A master mix of peptide-coupled beads was prepared in PBS-TBN buffer and 100 μL, of the bead master mix was added to filter plates. After washing the beads, 100 pt of subject's plasma at 1:10 dilution was added to the triplicate wells. The plates were incubated on a shaker for 2 hours at 300 rpm at room temperature. Excess plasma was removed and the plate was washed. 50 μL/well of mouse anti-human IgE-PE (Thermo-Pierce Antibodies, Clone BES, diluted 1:50 in PBS-TBN) or mouse anti-human IgG4 Fc-PE (SouthernBiotech, Clone HP6025, diluted 1:400 in PBS-TBN) secondary antibody was added and the plates were incubated for 30 minutes. After a final wash, 100 μL of PBS-TBN buffer was added to each well to re-suspend the beads, which were then transferred to fixed-bottom 96-well reading plates, and quantified on the Luminex 200 instrument (Luminex® 100/200™ System, Luminex Corporation, Austin, Tex.).

All samples were processed in triplicates. To eliminate background intensity, a buffer sample (PBS-TBN buffer) was also processed in triplicates in each plate. The MFI for each epitope and sample was obtained directly from the Luminex reader's output.

### Validation

Validation of the test was performed on 81 subjects (23 allergic, 58 non-allergic) from a CoFAR2 study. All diagnoses were determined by OFC at age 5 years. Plasma samples were obtained at years 2 and 5 for each subject. These samples were analyzed using BBEA methodology to obtain IgE and IgG4 epitope levels for each subject at year 2 and year 5. Luminex data was collected in triplicate for all subject samples as well as for the negative (buffer) and positive control. Data was processed in the following manner:
1) replicates were merged into a single value using the median function;
2) the subject, buffer, and positive controls values were all log transformed;
3) background subtraction was performed by eliminating the buffer value from all subject and positive control values; and
4) the median positive control value was determined for each plate, and this was used to calibrate all subject values on each plate according to its own positive control value.

Validation of the diagnostic test was performed using predefined hypotheses and thresholds. First, the performance of the diagnostic test using threshold 0.1 Ku/L for sIgE and threshold 0.30 for the optimal IgE (IgG4) epitope was statistically significant using the chi-squared test for association for subjects at year 5. Similarly, the performance of the diagnostic test for subjects at year 2 was assessed. All data analyses performed using Matlab R2015b.

### Results

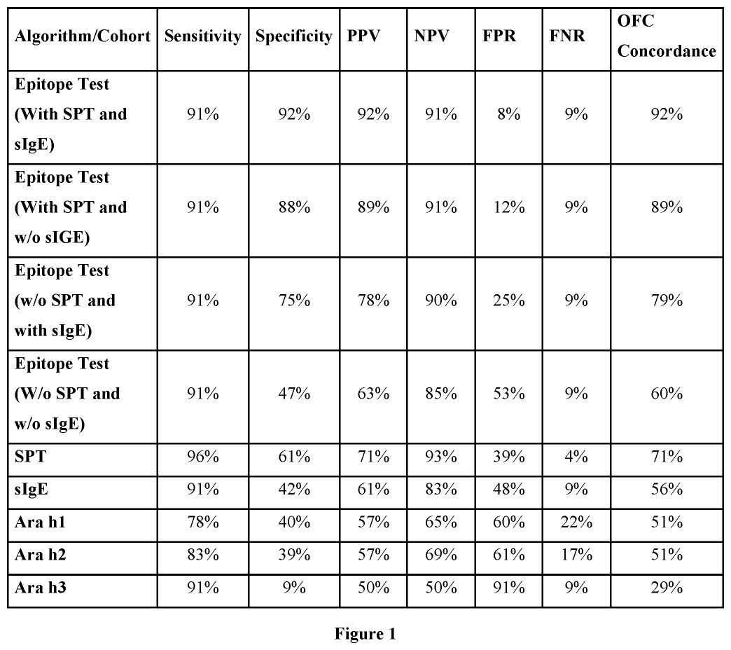

The results of the test on the validation cohort are displayed in FIG. 1. Performance of other diagnostic tests are displayed for comparison. The results for 10 allergic and 10 non-allergic subjects from the validation cohort appear in Table 1 below.

TABLE 1

| | Allergy | SPT | sIgE | h2. | h2. | Correct |
|---|---|---|---|---|---|---|
| Subject | Status | (mm) | (kU/L) | 008 | 019 | Call |
| 19 | Not Allergic | 7.5 | 0.31 | −0.39 | 0.99 | TRUE |
| 24 | Not Allergic | 4 | 3.88 | 0.94 | 0.70 | FALSE |
| 33 | Not Allergic | 6.5 | 11.17 | −0.05 | 1.81 | TRUE |
| 76 | Not Allergic | 7 | 4.41 | −0.44 | 3.22 | TRUE |
| 133 | Not Allergic | 16 | 1.59 | −0.10 | 2.02 | TRUE |
| 172 | Not Allergic | 5.5 | 0.21 | 0.34 | 1.22 | FALSE |
| 226 | Not Allergic | 4 | 1.12 | −0.06 | 1.98 | TRUE |
| 254 | Not Allergic | 3.5 | 4.79 | 0.10 | 1.07 | TRUE |
| 259 | Not Allergic | 15.5 | 5.3 | 2.90 | 1.96 | FALSE |
| 273 | Not Allergic | 14 | 4.06 | −1.05 | 5.42 | TRUE |
| 26 | Allergic | 13.5 | 7.95 | 9.60 | 2.52 | TRUE |
| 49 | Allergic | 14.5 | 3.67 | 7.87 | −0.04 | TRUE |
| 60 | Allergic | 11 | 11.7 | 10.94 | 16.25 | TRUE |
| 86 | Allergic | 9.5 | 0.74 | 1.49 | 2.53 | TRUE |
| 113 | Allergic | 4 | 0.34 | 0.45 | 1.00 | TRUE |
| 152 | Allergic | 4.5 | 6.8 | 0.25 | −0.68 | TRUE |
| 190 | Allergic | 14 | 0.49 | 0.91 | −2.25 | TRUE |
| 199 | Allergic | 14 | 0.6 | 0.58 | 1.42 | TRUE |
| 231 | Allergic | 11 | 7.25 | 1.77 | 1.17 | TRUE |
| 441 | Allergic | 8 | 11.24 | 3.18 | 5.21 | TRUE |

Raw data obtained from the epitope assay was reported as MFIs. All data was processed as described above.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method for diagnosing a peanut allergy in a subject comprising:
contacting two peanut peptides coupled to a solid support with a biological sample obtained from the subject under conditions sufficient to permit binding of one or more allergy associated immunoglobulins (AAIs) in the biological sample to the two peanut peptides to form AAI-peptide-solid support complexes, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein;
contacting the AAI-peptide-solid support complexes with an AAI-specific labeling reagent to form labeling reagent-AAI-peptide-solid support complexes; and
measuring the binding of the AAI-specific labeling reagent to each AAI-peptide-solid support complex;
wherein when the combined binding for each peanut peptide in the AAI-peptide solid support complex to the AAI-specific labeling reagent is greater than a threshold value, the subject is allergic to peanuts, and when the combined binding for each peanut peptide in the AAI-peptide solid support complex to the AAI-specific labeling reagent is equal to or less than the threshold value, the subject is not allergic to peanuts.

2. The method according to claim 1, wherein one of the two peanut peptides comprises the amino acid sequence according to SEQ ID NO:1, and the other of the two peanut peptides comprises the amino acid sequence according to SEQ ID NO:2.

3. The method according to claim 1, wherein the solid support is a microsphere bead, glass array, silicone array, membrane, or microtiter plate.

4. The method according to claim 1, wherein the AAI is IgG and/or IgE.

5. The method according to claim 1, wherein the AAI-specific labeling reagent is a detectably labeled anti-human antibody.

6. The method according to claim 1, wherein the measuring of the binding of the AAI-specific labeling reagent to each AAI-peptide-solid support complex is carried out by a point of care device.

7. The method according to claim 1, wherein measuring the binding of the AAI-specific labeling reagent to each AAI-peptide-solid support complex comprises measuring the mean fluorescent intensity (MFI) of each AAI-specific labeling reagent, wherein:
when h2.008+(h2.019)/20 is ≤0.20, the subject is not allergic to peanuts; and
when h2.008+(h2.019)/20 is >0.20, the subject is allergic to peanuts;
wherein h2.008 is the calibrated net MFI of the labeling reagent-AAI-peptide-solid support complex wherein the peanut peptide comprises SEQ ID NO:1, and h2.019 is the calibrated net MFI of the labeling reagent-AAI-peptide-solid support complex wherein the peanut peptide comprises SEQ ID NO:2; and
wherein the AAI-specific labeling reagent is a detectably labeled anti-human antibody.

8. The method according to claim 1, further comprising performing or having performed a skin prick test (SPT) and/or a total peanut specific IgE (sIgE) test.

9. The method according to claim 8, wherein when the SPT is ≤3 mm and/or the sIgE is ≤0.10 kU/L then the subject is not allergic to peanuts, and when the SPT is ≥18 mm and/or the sIgE is ≥18 kU/L then the subject is allergic to peanuts.

10. A method for detecting development of clinical tolerance to peanuts in a subject that is allergic to peanuts comprising:
contacting two peanut peptides coupled to a solid support with a biological sample obtained from the subject under conditions sufficient to permit binding of one or more allergy associated immunoglobulins (AAIs) in the biological sample to the two peanut peptides to form two AAI-peptide-solid support complexes, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein;
contacting the AAI-peptide-solid support complexes with an AAI-specific labeling reagent to form labeling reagent-AAI-peptide-solid support complexes;
measuring the binding of the AAI-specific labeling reagent to each AAI-peptide-solid support complex; and
comparing the binding of the AAI-specific labeling reagent to each AAI-peptide-solid support complex to the binding of the AAI-specific labeling reagent to each AAI-peptide-solid support complex from a biological sample previously obtained from the subject;
wherein when the combined binding for each peanut peptide in the AAI-peptide solid support complex to the AAI-specific labeling reagent is greater than or equal to the combined binding for each peanut peptide in the AAI-peptide solid support complex to the AAI-specific labeling reagent for the previously obtained biological sample, the subject has not established clinical tolerance to peanuts; and when the combined binding for each peanut peptide in the AAI-peptide solid support complex to the AM-specific labeling reagent is less than the combined binding for each peanut peptide in the AAI-peptide solid support complex to the AAI-specific labeling reagent for the previously obtained biological sample, the subject has established clinical tolerance to peanuts.

11. The method according to claim 10, wherein one of the two peanut peptides comprises the amino acid sequence according to SEQ ID NO:1, and the other of the two peanut peptides comprises the amino acid sequence according to SEQ ID NO:2.

12. The method according to claim 10, wherein the AAI is IgG and/or IgE.

13. The method according to claim 10, wherein the AAI-specific labeling reagent is a detectably labeled anti-human antibody.

14. The method according to claim 10, wherein the measuring of the binding of the AAI-specific labeling reagent to each AAI-peptide-solid support complex is carried out by a point of care device.

15. The method according to claim 10, wherein measuring the binding of the AAI-specific labeling reagent to each AAI-peptide-solid support complex comprises measuring the mean fluorescent intensity (MFI) of each AAI-specific labeling reagent, wherein:
when h2.008+(h2.019)/20 is ≤0.20, the subject is not allergic to peanuts; and
when h2.008+(h2.019)/20 is >0.20, the subject is allergic to peanuts;
wherein h2.008 is the calibrated net MFI of the labeling reagent-AAI-peptide-solid support complex wherein the peanut peptide comprises SEQ ID NO:1, and h2.019 is the calibrated net MFI of the labeling reagent-AAI-peptide-solid support complex wherein the peanut peptide comprises SEQ ID NO:2; and
wherein the AAI-specific labeling reagent is a detectably labeled anti-human antibody.

16. A method of desensitizing an infant to two peanut allergens to induce tolerance or non-allergy to peanuts comprising administering two peanut peptides to the infant, wherein one of the two peanut peptides comprises the amino acid sequence WELQGDRRCQSQLER (SEQ ID NO:1) or an amino acid sequence comprising SEQ ID NO:1 but having one to four conservative amino acid substitutions therein, and the other of the two peanut peptides comprises the amino acid sequence DSYERDPYSPSQDPY (SEQ ID NO:2) or an amino acid sequence comprising SEQ ID NO:2 but having one to four conservative amino acid substitutions therein.

17. The method according to claim 16, wherein one of the two peanut peptides comprises the amino acid sequence according to SEQ ID NO:1, and the other of the two peanut peptides comprises the amino acid sequence according to SEQ ID NO:2.

18. A composition consisting of a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein.

19. The composition according to claim 18, wherein the first peanut peptide comprises the amino acid sequence according to SEQ ID NO:1, and the second peanut peptide comprises the amino acid sequence according to SEQ ID NO:2.

20. A kit comprising:
a solid support coupled to a first peanut peptide comprising the amino acid sequence WELQGDRRCQSQLER (SEQ ID NO:1) or an amino acid sequence comprising SEQ ID NO:1 but having one to four conservative amino acid substitutions therein, and a second peanut peptide comprising the amino acid sequence DSYERDPYSPSQDPY (SEQ ID NO:2) or an amino acid sequence comprising SEQ ID NO:2 but having one to four conservative amino acid substitutions therein; and
an allergy associated immunoglobulin (AAI)-specific labeling reagent.
